(19) United States
(12) Patent Application Publication
(10) Pub. No.: US 2018/0030509 A1
(43) Pub. Date: Feb. 1, 2018

(54) NUCLEIC ACID AMPLIFICATION REACTION METHOD, REAGENT, AND METHOD OF USING REAGENT

(71) Applicant: Seiko Epson Corporation, Tokyo (JP)

(72) Inventors: Masato HANAMURA, Shiojiri (JP); Kotaro IDEGAMI, Chino (JP); Masayuki UEHARA, Matsumoto (JP)

(21) Appl. No.: 15/650,114

(22) Filed: Jul. 14, 2017

(30) Foreign Application Priority Data
Jul. 29, 2016 (JP) 2016-149430
Mar. 15, 2017 (JP) 2017-049610

(51) Int. Cl. C12Q 1/68; G01N 1/40; G01N 27/447

(52) U.S. Cl. CPC C12Q 1/686; C12Q 1/6806; G01N 27/447; C12Q 1/6853; G01N 1/40; G01N 2001/4038; C12Q 1/00

(57) ABSTRACT

A nucleic acid amplification reaction method includes a heating step of heating a reaction solution containing a reverse transcriptase, a polymerase, a primer, and a probe for performing a reverse transcription reaction, and a thermal cycling step of performing thermal cycling for amplifying a nucleic acid for the reaction solution after the heating step, wherein the Tm value of the primer is 65° C. or higher and 80° C. or lower, and in the heating step, a heating time for the reaction solution is 5 seconds or more and 480 seconds or less.

### BACKGROUND

### 1. Technical Field

The present invention relates to a nucleic acid amplification reaction method, a reagent, and a method of using a reagent.

### 2. Related Art

In recent years, due to the development of technologies utilizing genes, medical treatments utilizing genes such as gene diagnosis or gene therapy have been drawing attention. In addition, many methods using genes in determination of breed varieties or breed improvement have also been developed in agriculture and livestock industries. As technologies for utilizing genes, technologies such as a PCR (Polymerase Chain Reaction) method are widely used. Nowadays, the PCR method has become an indispensable technology for elucidation of information on biological materials.

The PCR method is a method of amplifying a target nucleic acid by performing thermal cycling for a solution (reaction solution) containing a nucleic acid to be amplified (target nucleic acid) and a reagent. The thermal cycling is a treatment of periodically subjecting the reaction solution to two or more temperature steps. In the PCR method, a method of performing two- or three-step thermal cycling is generally used.

An increase in PCR speed is a necessary technology for reducing the testing time of a genetic test, and has been much expected in the genetic testing industries.

For example, JP-T-2015-520614 (Patent Document 1) discloses a method in which a polymerase is provided at a concentration of at least 0.5 μM and a primer is provided at a concentration of at least 2 μM, and a cycle is completed in a cycle time of less than 20 seconds per cycle.

Meanwhile, there are largely two types of nucleic acids: DNA (deoxyribonucleic acid); and RNA (ribonucleic acid). In the case where a template nucleic acid is an RNA, the RNA is reverse-transcribed into a DNA by a reverse transcription reaction (reverse transcription) prior to PCR.

In a heating step for a reverse transcription reaction as described above and a thermal cycling step of performing thermal cycling for nucleic acid amplification, the same primer can be used. As a result of intensive studies, the present inventors found that the condition for suppressing nonspecific amplification in the reverse transcription reaction varies when a desired primer is selected for trying to increase the thermal cycling speed.

### SUMMARY

An advantage of some aspects of the invention is to provide a nucleic acid amplification reaction method capable of suppressing amplification of a nonspecific nucleic acid. Another advantage of some aspects of the invention is to provide a reagent capable of suppressing amplification of a nonspecific nucleic acid, and a method of using the same.

A nucleic acid amplification reaction method according to an aspect of the invention includes a heating step of heating a reaction solution containing a reverse transcriptase, a polymerase, a primer, and a probe for performing a reverse transcription reaction, and a thermal cycling step of performing thermal cycling for amplifying a nucleic acid for the reaction solution after the heating step, wherein the Tm value of the primer is 65° C. or higher and 80° C. or lower, and in the heating step, a heating time for the reaction solution is 5 seconds or more and 480 seconds or less.

Such a nucleic acid amplification reaction method is a nucleic acid amplification reaction method capable of performing a reverse transcription reaction and also increasing the thermal cycling speed, and can suppress amplification of a nonspecific nucleic acid (see the below-mentioned “3. Experimental Examples” for the details).

In the nucleic acid amplification reaction method according to the aspect of the invention, in the heating step, the reaction solution may be heated to 50° C. or higher and 70° C. or lower.

According to such a nucleic acid amplification reaction method, amplification of a nonspecific nucleic acid can be suppressed.

In the nucleic acid amplification reaction method according to the aspect of the invention, in the heating step, a heating time for the reaction solution may be 10 seconds or more and 60 seconds or less.

According to such a nucleic acid amplification reaction method, in the measurement of a fluorescence intensity, the fluorescence intensity can be increased (see the below-mentioned “3. Experimental Examples” for the details).

In the nucleic acid amplification reaction method according to the aspect of the invention, the amount of the reverse transcriptase contained in the reaction solution may be 30 units or more and 60 units or less.

According to such a nucleic acid amplification reaction method, in the measurement of a fluorescence intensity, the fluorescence intensity can be increased (see the below-mentioned “3. Experimental Examples” for the details), and also the cost can be reduced.

In the nucleic acid amplification reaction method according to the aspect of the invention, in the heating step, the reaction solution may be heated to 60° C. or higher and 70° C. or lower, and a heating time for the reaction solution may be 10 seconds or more and 30 seconds or less.

According to such a nucleic acid amplification reaction method, while ensuring a fluorescence intensity, a reverse transcription reaction time can be reduced (see the below-mentioned “3. Experimental Examples” for the details).

In the nucleic acid amplification reaction method according to the aspect of the invention, in the heating step, the reaction solution may be heated to 66° C. or higher and 70° C. or lower, and a heating time for the reaction solution may be 60 seconds or less.

According to such a nucleic acid amplification reaction method, in the measurement of a fluorescence intensity, the fluorescence intensity can be further increased (see the below-mentioned “3. Experimental Examples” for the details).

In the nucleic acid amplification reaction method according to the aspect of the invention, in the thermal cycling step, the time per cycle of the thermal cycling may be 9 seconds or less.

According to such a nucleic acid amplification reaction method, the thermal cycling speed can be increased.

In the nucleic acid amplification reaction method according to the aspect of the invention, a heating time for an annealing reaction for the primer may be 6 seconds or less.

According to such a nucleic acid amplification reaction method, the thermal cycling speed can be increased.

In the nucleic acid amplification reaction method according to the aspect of the invention, the reaction solution may contain a divalent cation, and the concentration of the divalent cation contained in the reaction solution may be 2 mM or more and 7.5 mM or less.

According to such a nucleic acid amplification reaction method, while accelerating an elongation reaction by a polymerase and increasing the PCR speed, nonspecific amplification is suppressed, and a decrease in yield of a specific amplification product can be suppressed.

In the nucleic acid amplification reaction method according to the aspect of the invention, the reaction solution may contain MgCl₂, the divalent cation may be derived from MgCl₂, and the concentration of MgCl₂ contained in the reaction solution may be 4 mM or more and 7.5 mM or less.

According to such a nucleic acid amplification reaction method, while accelerating an elongation reaction by a polymerase and increasing the thermal cycling speed, a decrease in yield of a specific amplification product due to an increase in nonspecific amplification because of too much Mg²⁺ can be prevented from occurring.

In the nucleic acid amplification reaction method according to the aspect of the invention, the reaction solution may contain MgSO₄, the divalent cation may be derived from MgSO₄, and the concentration of MgSO₄ contained in the reaction solution may be 2 mM or more and 3 mM or less.

According to such a nucleic acid amplification reaction method, while accelerating an elongation reaction by a polymerase and increasing the thermal cycling speed, a decrease in yield of a specific amplification product due to an increase in nonspecific amplification because of too much Mg²⁺ can be prevented from occurring.

In such a nucleic acid amplification reaction method, an optimal concentration range of the divalent cation for suppressing nonspecific amplification and suppressing a decrease in yield of a specific amplification product while accelerating an elongation reaction and increasing the PCR speed varies depending on the type of the divalent cation.

In the nucleic acid amplification reaction method according to the aspect of the invention, the probe may be a hydrolysis probe.

According to such a nucleic acid amplification reaction method, when the probe is degraded by the polymerase, a quenching effect is cancelled, and a reporter dye emits light, whereby the amplification amount of a nucleic acid can be quantitatively determined.

In the nucleic acid amplification reaction method according to the aspect of the invention, the probe may contain at least one of an artificial nucleic acid and a minor groove binder molecule.

According to such a nucleic acid amplification reaction method, the Tm value of the probe can be increased while suppressing an increase in the number of bases of the probe.

In the nucleic acid amplification reaction method according to the aspect of the invention, the reverse transcriptase may be derived from mouse Moloney murine leukemia virus.

According to such a nucleic acid amplification reaction method, amplification of a nonspecific nucleic acid can be suppressed.

In the nucleic acid amplification reaction method according to the aspect of the invention, the primer may contain an artificial nucleic acid.

According to such a nucleic acid amplification reaction method, amplification of a nonspecific nucleic acid can be suppressed (see the below-mentioned “3. Experimental Examples” for the details).

In the nucleic acid amplification reaction method according to the aspect of the invention, the primer may be a sequence-specific primer for a target RNA.

According to such a nucleic acid amplification reaction method, the primer anneals only to a specific base sequence, and therefore, amplification of a nonspecific nucleic acid can be suppressed.

A reagent according to an aspect of the invention is a reagent for performing a reverse transcription reaction and a nucleic acid amplification reaction, and includes a reverse transcriptase, a polymerase, a primer, a probe, and MgCl₂, wherein the Tm value of the primer is 65° C. or higher and 80° C. or lower, and when the reagent becomes a reaction solution for performing a reverse transcription reaction and a nucleic acid amplification reaction, the concentration of MgCl₂ contained in the reaction solution is 4 mM or more and 7.5 mM or less.

Such a reagent can be used in a nucleic acid amplification reaction method capable of performing a reverse transcription reaction and also increasing the thermal cycling speed, and can suppress amplification of a nonspecific nucleic acid.

A reagent according to an aspect of the invention is a reagent for performing a reverse transcription reaction and a nucleic acid amplification reaction, and includes a reverse transcriptase, a polymerase, a primer, a probe, and MgSO₄, wherein the Tm value of the primer is 65° C. or higher and 80° C. or lower, and when the reagent becomes a reaction solution for performing a reverse transcription reaction and a nucleic acid amplification reaction, the concentration of MgSO₄ contained in the reaction solution is 2 mM or more and 3 mM or less.

Such a reagent can be used in a nucleic acid amplification reaction method capable of performing a reverse transcription reaction and also increasing the thermal cycling speed, and can suppress amplification of a nonspecific nucleic acid.

A method of using a reagent according to an aspect of the invention is a method of using the reagent according to the aspect of the invention, including preparing the reaction solution by bringing the reagent and a template nucleic acid solution containing a template nucleic acid into contact with each other, performing reverse transcription reaction by heating the reaction solution for 5 seconds or more and 480 seconds or less, and performing, after the performing reverse transcription reaction, performing a nucleic acid amplification reaction by performing thermal cycling for the reaction solution.

Such a method of using a reagent can be used in a nucleic acid amplification reaction method capable of performing a reverse transcription reaction and also increasing the thermal cycling speed, and can suppress amplification of a nonspecific nucleic acid.

### BRIEF DESCRIPTION OF THE DRAWINGS

The invention will be described with reference to the accompanying drawings, wherein like numbers reference like elements.

FIG. 1 is a graph showing a relationship between a temperature for Taq polymerase and a relative activity efficiency.

FIG. 2 is a flowchart for illustrating a nucleic acid amplification reaction method according to an embodiment.

FIG. 3 is a cross-sectional view schematically showing a thermal cycler for performing thermal cycling for a reaction solution according to an embodiment.

FIG. 4 is a graph showing a relationship between a heating time for a reverse transcription reaction and a fluorescence intensity.

FIG. 5 is a graph showing a relationship between a heating time for a reverse transcription reaction and a fluorescence intensity.

FIG. 6 is a graph showing a relationship between a heating time for a reverse transcription reaction and a relative fluorescence intensity.

FIG. 7 is a graph showing a relationship between a heating time for a reverse transcription reaction and a relative fluorescence intensity.

FIG. 8 shows the results of electrophoresis.

FIG. 9 is a graph showing a relationship between a heating time for a reverse transcription reaction and a fluorescence intensity.

FIG. 10 is a graph showing a relationship between a heating time for a reverse transcription reaction and a fluorescence intensity.

FIG. 11 is a graph showing a relationship between the amount of a reverse transcriptase and a fluorescence intensity.

FIG. 12 is a graph showing a relationship between a heating time for a reverse transcription reaction and a fluorescence intensity.

FIG. 13 is a graph showing a relationship between a heating time for a reverse transcription reaction and a fluorescence intensity.

FIG. 14 is a graph showing a relationship between a heating time for a reverse transcription reaction and a fluorescence intensity.

FIG. 15 is a graph showing a relationship between a PCR reaction time and a fluorescence intensity.

FIG. 16 is a graph showing a relationship between a PCR reaction time and a fluorescence intensity.

FIG. 17 is a graph showing a relationship between a reverse transcription reaction time and a fluorescence intensity.

FIG. 18 shows the results of electrophoresis.

FIG. 19 shows the results of electrophoresis.

### DESCRIPTION OF EXEMPLARY EMBODIMENTS

Hereinafter, preferred embodiments of the invention will be described in detail with reference to the accompanying drawings. Note that the embodiments described below are not intended to unduly limit the content of the invention described in the appended claims. Further, all the configurations described below are not necessarily essential components of the invention.

### 1. REAGENT

First, a reagent according to this embodiment will be described. The reagent according to this embodiment is a reagent for performing a reverse transcription reaction and a nucleic acid amplification reaction. The reagent may be, for example, in a liquid form or may be in a lyophilized state. For example, the reagent in a lyophilized state is fixed in a container (not shown), and a template nucleic acid solution containing an RNA is introduced into the container so as to bring the template nucleic acid solution and the reagent into contact with each other. The reagent in a lyophilized state is dissolved in the aqueous component of the template nucleic acid solution and incorporated into the template nucleic acid solution so as to become a reaction solution. Therefore, the reaction solution contains the template nucleic acid and the reagent, and thus serves as a place for allowing a nucleic acid amplification reaction to proceed.

The reagent contains a primer, a polymerase, a probe, dNTP, a buffer, and a reverse transcriptase. The reagent according to this embodiment is, for example, a reagent for one-step reverse transcription-PCR in which a reverse transcription reaction and a nucleic acid amplification reaction (PCR) are continuously performed.

### 1.1. Primer

The primer is designed to anneal to a template nucleic acid (template). The “anneal (annealing)” refers to an action (a phenomenon) in which a primer binds to a DNA. The reagent contains a forward primer which anneals to one template nucleic acid having a single-stranded structure (single-stranded DNA) after a template nucleic acid having a double-stranded structure (double-stranded DNA) is denatured, and a reverse primer which anneals to the other single-stranded DNA as the primer. The concentrations of the forward primer and the reverse primer contained in the reaction solution are each, for example, 0.4 μM or more and 6.4 μM or less, preferably 0.8 μM or more and 3.2 μM or less. The concentration of the forward primer and the concentration of the reverse primer contained in the reaction solution may be the same as or different from each other.

The Tm value of the primer (the forward primer and the reverse primer) is 65° C. or higher and 80° C. or lower, preferably 70° C. or higher and 75° C. or lower. According to this, the reagent according to this embodiment can increase the thermal cycling speed (can reduce the thermal cycling time) (see the below-mentioned “3. Experimental Examples” for the details). The Tm value is an index of the temperature at which a primer anneals to a template nucleic acid, and is a temperature at which 50% of a double-stranded DNA is dissociated into single-stranded DNAs, that is, a melting temperature. If the temperature is not lower than the Tm value, not less than half of the primer anneals to the template nucleic acid. The Tm value of the forward primer and the Tm value of the reverse primer may be the same as or different from each other.

As a calculation method of the Tm value, for example, a nearest neighbor method is exemplified, and the Tm value can be calculated according to the following formula (1).

Tm=1000 ΔH/(−10.8+ΔS+R×lm(Ct/4))−273.15+16.6 [Na⁺] (1)

In the formula (1), ΔH represents the sum (kcal/mol) of the nearest neighbor enthalpy changes for hybrids, ΔS represents the sum (cal/mol/K) of the nearest neighbor entropy changes for hybrids, R represents the gas constant (1.987 cal/deg/mol), Ct represents the molar concentration (mol/L) of the primer, and Na⁺ represents the concentration (mol/L) of a monovalent cation contained in the buffer.

In the case where the Tm value of the primer is increased by increasing the number of bases of the primer, by designing the primer so as to be elongated inside the amplification region (so that the primer is elongated on the 5′-end side of the template nucleic acid), the Tm value can be increased without increasing the amplification region of a nucleic acid by the elongation reaction. According to this, the thermal cycling speed can be increased.

The primer may contain an artificial nucleic acid. According to this, amplification of a nonspecific nucleic acid can be suppressed. The artificial nucleic acid will be described in the below-mentioned “1.4. Probe”.

The primer may be a sequence-specific primer for a target RNA (an RNA to which the primer anneals). That is, the primer may be a primer which anneals only to a specific base sequence of the target RNA. According to this, the primer anneals only to a specific base sequence, and therefore, amplification of a nonspecific nucleic acid can be suppressed. The sequence-specific primer for a target RNA is effective in the case where the sequence of the target RNA is known.

### 1.2. Polymerase

The polymerase is not particularly limited, however, examples thereof include a DNA polymerase. The DNA polymerase polymerizes nucleotides complementary to the bases of a template nucleic acid at the end of the primer annealing to the template nucleic acid having a single-stranded structure (single-stranded DNA). The DNA polymerase is preferably a heat-resistant enzyme or an enzyme for PCR, and there are a large number of commercially available products, for example, Taq polymerase, KOD polymerase, Tfi polymerase, Tth polymerase, modified forms thereof, and the like, however, a DNA polymerase capable of performing hot start is preferred. As the polymerase, there are a hydrolysis-type polymerase which degrades a probe by hydrolysis such as Taq polymerase, and a non-hydrolysis-type polymerase which does not degrade a probe by hydrolysis such as KOD polymerase. The KOD polymerase is derived from Thermococcus kodakarensis KOD1 and is a DNA polymerase from the genus Thermococcus. The amount of the polymerase contained in the reaction solution is, for example, 0.5 units (U) or more.

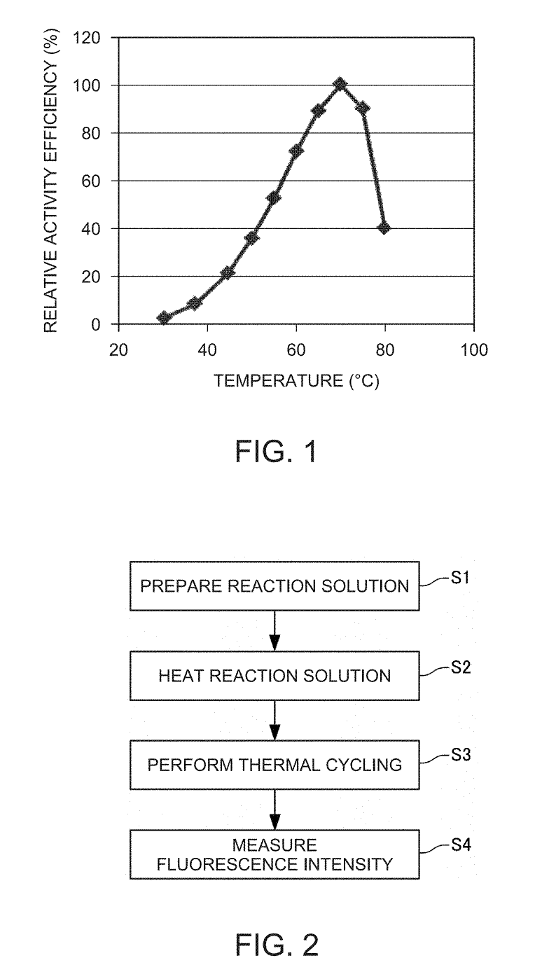

FIG. 1 is a graph showing a relationship between a temperature for Taq polymerase and a relative activity efficiency. The vertical axis represents a relative activity efficiency when the maximum value of the activity efficiency of the polymerase which is reached while changing the temperature is assumed to be 100%. The activity efficiency of the polymerase at each temperature can be obtained by performing a procedure so that dNTP emits light when it is incorporated during the elongation reaction, and measuring the fluorescence intensity (fluorescent brightness) from the dNTP after a predetermined period of time has elapsed. As the fluorescence intensity is higher, the activity efficiency of the polymerase is higher. In the example shown in FIG. 1, the relative activity efficiency of the Taq polymerase reached the maximum when the temperature was around 70° C.

### 1.3. dNTP

The dNTP refers to a mixture of four types of deoxyribonucleotide triphosphates. That is, the dNTP refers to a mixture of dATP, dCTP, dGTP, and dTTP. The DNA polymerase forms a new DNA by joining dATP, dCTP, dGTP, or dTTP to the end of the primer annealing to the template (an elongation reaction). The concentration of the dNTP contained in the reaction solution is, for example, 0.06 mM or more and 0.75 mM or less, preferably 0.125 mM or more and 0.5 mM or less.

1.4. Probe

The probe is a fluorescently labeled probe to be used for quantitatively determining the amplification amount of a nucleic acid. The concentration of the probe contained in the reaction solution is 0.5 μM or more and 2.4 μM or less, preferably 0.5 μM or more and 1.8 μM or less.

The probe is, for example, a hydrolysis probe containing a reporter dye and a quencher dye. More specifically, the probe is TaqMan (registered trademark) probe. While the hydrolysis probe hybridizes to a single-stranded DNA to form a double-stranded structure, the light emission of a reporter dye is suppressed by a quencher dye (by a quenching effect) which is in close proximity to the reporter dye. However, when the probe is degraded by the exonuclease activity of the polymerase, the quenching effect is cancelled, and therefore, the reporter dye emits light. By this light emission, the amplification amount of a nucleic acid can be quantitatively determined. The “hybridization” refers to a phenomenon in which a probe binds to a DNA. In the case where a hydrolysis probe is used as the probe, Taq polymerase is used as the polymerase.

The probe may be a non-hydrolysis probe other than the hydrolysis probe. Specifically, the probe may be a Q (Quenching) probe utilizing a fluorescence-quenching phenomenon. The Q probe emits light in a state where it does not hybridize to a single-stranded DNA, and quenches the light when it hybridizes to a single-stranded DNA. By this difference in the emission intensity, the amplification amount of a nucleic acid can be quantitatively determined. In the case where the Q probe is used as the probe, KOD polymerase is used as the polymerase. The elongation reaction rate of KOD polymerase is larger than that of Taq polymerase, and therefore, KOD polymerase can increase the thermal cycling speed.

In the case where the probe is a non-hydrolysis probe, it is not necessary to degrade the probe in the elongation reaction, and therefore, there is no need to provide an amplification region to which the probe anneals between the forward primer and the reverse primer. According to this, it becomes possible to design the amplification region narrower than in a hydrolysis-type system. Since the amplification region becomes narrower, the annealing time can be reduced, and thus, the thermal cycling speed can be increased.

The probe may contain at least one of an artificial nucleic acid and a minor groove binder (MGB) molecule. According to this configuration, the Tm value of the probe can be increased while suppressing an increase in the number of bases of the probe (while suppressing an increase in the base length). When the number of bases of the probe is increased, for example, a time for degrading the probe is increased, and therefore, it is sometimes difficult to increase the PCR speed.

The “artificial nucleic acid” refers to a nucleic acid molecule which can bind to a base of a DNA or an RNA through a hydrogen bond and is other than natural nucleic acid molecules. Examples of the artificial nucleic acid include a 2′,4′-BNA (2′-O,4′-C-methano-bridged nucleic acid, also known as “LNA (Locked Nucleic Acid)”) in which the oxygen atom at the 2′-position of a ribose ring of a nucleic acid is methylene-crosslinked to the carbon atom at the 4′-position. The chemical formula of the LNA is shown in the following formula (2).

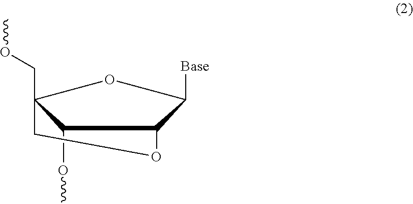

In the formula (2), examples of the base include T (thymine), C (cytosine), G (guanine), and A (adenine), but are not particularly limited. Further, the base may be a base modified by methylation, acetylation, or the like.

The artificial nucleic acid may be an LNA analog obtained by modifying an LNA, and specifically may be 3′-amino-2′,4′-BNA, 2′,4′-BNA^{COC}, or 2′,4′-BNA^{NC} (N-Me). Further, an artificial nucleic acid contained in a modified fluorescent probe may be a PNA (Peptide Nucleic Acid), a GNA (Glycol Nucleic Acid), a TNA (Threose Nucleic Acid), or an analog obtained by modifying such a molecule. The number of artificial nucleic acids contained in the probe is not particularly limited, and one probe may contain a plurality of artificial nucleic acids.

### 1.5. Buffer

The buffer is, for example, a buffer agent containing a salt. Examples of the salt contained in the buffer include salts such as Tris, HEPES, PIPES, and phosphates. By using such a salt, the pH of the buffer can be adjusted.

The buffer contains a divalent cation. Examples of the divalent cation include Mn²⁺, Co²⁺, and Mg²⁺. In the case where the nucleic acid amplification reaction reagent becomes a reaction solution for performing a nucleic acid amplification reaction, the concentration of the divalent cation contained in the reaction solution is 2 mM or more and 7.5 mM or less. By setting the concentration of the divalent cation to 2 mM or more, the elongation reaction by the polymerase is accelerated, and the PCR speed can be increased (specifically, the time per cycle of the thermal cycling can be reduced to 9 seconds or less). By setting the concentration of the divalent cation to 7.5 mM or less, nonspecific amplification is suppressed, and a decrease in yield of a specific amplification product can be suppressed.

Specifically, the buffer contains a divalent cationic compound, KCl, and Tris. More specifically, the buffer contains MgCl₂, and the divalent cation is derived from MgCl₂. That is, the divalent cation is produced by ionization of MgCl₂. In the case where the divalent cation is derived from MgCl₂, the concentration of Mg²⁺ is attributed to the activity of the polymerase. In the case where the nucleic acid amplification reaction reagent becomes a reaction solution for performing a nucleic acid amplification reaction, the concentration of MgCl₂ contained in the reaction solution is 4 mM or more and 7.5 mM or less, preferably 5 mM or more and mM or less, more preferably 5 mM. By setting the concentration of MgCl₂ to 4 mM or more, the elongation reaction by the polymerase is accelerated, and the PCR speed can be increased. By setting the concentration of MgCl₂ to 7.5 mM or less, nonspecific amplification is suppressed, and a decrease in yield of a specific amplification product can be suppressed. When the nucleic acid amplification reaction reagent is in a lyophilized state, the nucleic acid amplification reaction reagent is in a solid state, and contains MgCl₂, KCl, Tris, and an excipient such as trehalose.

The divalent cationic compound may be derived from MgSO₄. In this case, the buffer contains MgSO₄ in place of MgCl₂, and in the case where the nucleic acid amplification reaction reagent becomes a reaction solution for performing a nucleic acid amplification reaction, the concentration of MgSO₄ contained in the reaction solution is 2 mM or more and 3 mM or less, more preferably 2 mM. By setting the concentration of MgSO₄ to 2 mM or more, the elongation reaction by the polymerase is accelerated, and the PCR speed can be increased. By setting the concentration of MgSO₄ to 3 mM or less, nonspecific amplification is suppressed, and a decrease in yield of a specific amplification product can be suppressed.

### 1.6. Reverse Transcriptase

The reverse transcriptase is an enzyme that synthesizes a DNA using the base sequence of an RNA as a template. The reverse transcription reaction is a reaction in which a DNA is synthesized using the base sequence of an RNA as a template. In the reverse transcription reaction, by allowing one of the forward primer and the reverse primer to be used in PCR is allowed to anneal to an RNA, a DNA complementary to the RNA can be synthesized.

As the reverse transcriptase, for example, a reverse transcriptase derived from avian myeloblast virus, Ras-associated virus type 2, mouse Moloney murine leukemia virus, or human immunodefficiency virus type 1 is used.

The amount of the reverse transcriptase contained in the reaction solution is, for example, 5 units or more and 60 units or less, preferably 30 units or more and 60 units or less. By setting the amount of the reverse transcriptase contained in the reaction solution to 5 units or more, it is possible to suppress insufficient reverse transcription due to a too small amount of the reverse transcriptase. By setting the amount of the reverse transcriptase contained in the reaction solution to 60 units or less, the amount of the reverse transcriptase which is expensive can be decreased, and therefore, the cost can be reduced.

### 1.7. Other Components

In the case where the reagent is lyophilized, the reagent (lyophilized reagent) contains a sugar. Examples of the sugar include sucrose, trehalose, raffinose, and melezitose, each of which is a non-reducing sugar, among disaccharides and trisaccharides. Among the disaccharides and trisaccharides, particularly trehalose is preferably used because the function as a cryoprotective agent is high. Trehalose prevents the lyophilized reagent from coming into contact with a water molecule by its strong hydration force, and thus can improve the storage stability of the lyophilized reagent. The lyophilized reagent can be prepared by lyophilizing a mixed reagent solution containing the respective components of the reagent and a sugar. The temperature during lyophilization is, for example, about −80° C.

### 1.8. Using Method

In the method of using the reagent according to this embodiment, a reaction solution is prepared by bringing the reagent and a template nucleic acid solution containing a template nucleic acid into contact with each other, and after a reverse transcription reaction is performed by heating the reaction solution for 5 seconds or more and 480 seconds or less, a nucleic acid amplification reaction is performed by performing thermal cycling for the reaction solution.

The reagent according to this embodiment has, for example, the following characteristics.

In the reagent, a reverse transcriptase, a polymerase, a primer, a probe, and MgCl₂ are contained, the Tm value of the primer is 65° C. or higher and 80° C. or lower, and when the reagent becomes a reaction solution for performing a reverse transcription reaction and a nucleic acid amplification reaction, the concentration of MgCl₂ contained in the reaction solution is 4 mM or more and 7.5 mM or less. Further, in the reagent, a reverse transcriptase, a polymerase, a primer, a probe, and MgSO₄ are contained, the Tm value of the primer is 65° C. or higher and 80° C. or lower, and when the reagent becomes a reaction solution for performing a reverse transcription reaction and a nucleic acid amplification reaction, the concentration of MgSO₄ contained in the reaction solution is 2 mM or more and 3 mM or less. Therefore, the reagent can be used in a nucleic acid amplification reaction method capable of performing a reverse transcription reaction and also increasing the thermal cycling speed, and can suppress amplification of a nonspecific nucleic acid (which means that a primer anneals to a region other than a target region and a nucleic acid is amplified) (see the below-mentioned “3. Experimental Examples” for the details).

In the reagent, the probe may be a hydrolysis probe. Therefore, according to the reagent, in PCR, when the probe is degraded by the polymerase, a quenching effect is cancelled, and a reporter dye emits light, whereby the amplification amount of a nucleic acid can be quantitatively determined.

In the reagent, the probe may contain at least one of an artificial nucleic acid and an MGB molecule. Therefore, according to the reagent, the Tm value of the probe can be increased while suppressing an increase in the number of bases of the probe.

### 2. NUCLEIC ACID AMPLIFICATION REACTION METHOD

Next, the nucleic acid amplification reaction method according to this embodiment will be described with reference to the accompanying drawings. FIG. 2 is a flowchart for illustrating the nucleic acid amplification reaction method according to this embodiment.

First, a reaction solution is prepared by bringing the reagent according to this embodiment and a template nucleic acid solution into contact with each other (Step S1). Specifically, a template nucleic acid solution is introduced using a pipette or the like into a container in which the reagent is placed so as to bring the reagent and the template nucleic acid solution into contact with each other, whereby a reaction solution is prepared. The reaction solution contains, for example, a template nucleic acid, a primer, a probe, a polymerase, dNTP, a buffer, and a reverse transcriptase.

The template nucleic acid solution is obtained, for example, as follows. That is, a specimen, for example, a cell derived from an organism such as a human or a bacterium, a virus, or the like is collected using a collecting tool such as a cotton swab, and a template nucleic acid is extracted from the specimen using a known extraction method. Thereafter, a template nucleic acid solution is purified so as to have a predetermined concentration using a known purification method. The solution in the template nucleic acid solution is, for example, water (distilled water or sterile water) or a Tris-EDTA (ethylenediaminetetraacetic acid) (TE) solution.

Subsequently, the reaction solution is heated for performing a reverse transcription reaction (Step S2). In this step, the reaction solution is heated to, for example, 50° C. or higher and 70° C. or lower, preferably 60° C. or higher and 70° C. or lower, more preferably 66° C. or higher and 70° C. or lower. By heating the reaction solution to 50° C. or higher and 70° C. or lower, in the measurement of a fluorescence intensity after a nucleic acid is amplified (after a nucleic acid amplification reaction), the fluorescence intensity can be increased. A method for heating the reaction solution is not particularly limited, and for example, a method in which a beaker (water tank) is placed on a heat block or a hot plate, and a liquid (an aqueous sodium chloride solution or an oil) in the beaker is heated, and the container in which the reaction solution is contained is placed in the liquid, whereby the reaction solution is heated, and the like can be exemplified.

In the heating step for performing the reverse transcription reaction (Step S2), a heating time for the reaction solution is 5 seconds or more and 480 seconds or less, preferably 10 seconds or more and 60 seconds or less, more preferably 10 seconds or more and 30 seconds or less. By setting the heating time for the reaction solution to 5 seconds or more and 480 seconds or less, in the measurement of a fluorescence intensity after a nucleic acid is amplified, the fluorescence intensity can be increased (see the below-mentioned “3. Experimental Examples” for the details), and the amplification of a nucleic acid can be detected with high sensitivity. In this step, the “heating time for the reaction solution” is a time which does not include a time for increasing the temperature to a desired temperature for performing the reverse transcription reaction.

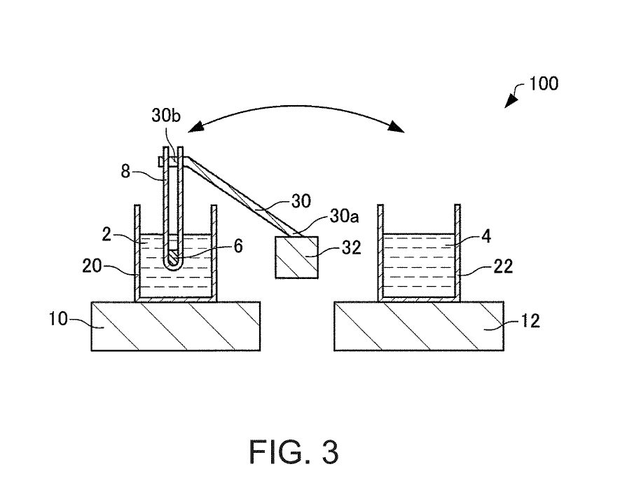

Subsequently, thermal cycling (for PCR) for amplifying a nucleic acid is performed for the reaction solution (Step S3). Here, FIG. 3 is a cross-sectional view schematically showing a thermal cycler 100 for performing thermal cycling for a reaction solution 6 according to this embodiment.

As shown in FIG. 3, the thermal cycler 100 includes a first hot plate 10, a second hot plate 12, a first beaker 20, a second beaker 22, an arm 30, and a fixing section 32.

The first hot plate 10 heats a liquid 2 contained in the first beaker 20 to a first temperature. The first temperature is a temperature suitable for the dissociation (denaturation reaction) of a double-stranded DNA, and is, for example, 85° C. or higher and 105° C. or lower. The liquid 2 is not particularly limited as long as it can be heated to the first temperature by the first hot plate 10, and for example, an aqueous sodium chloride solution and an oil can be exemplified.

The second hot plate 12 heats a liquid 4 contained in the second beaker 22 to a second temperature. The second temperature is lower than the first temperature. The second temperature is a temperature suitable for an annealing reaction and an elongation reaction, and is, for example, 55° C. or higher and 75° C. or lower. That is, in this step, in the heating for the annealing reaction for the primer, the elongation reaction is performed. That is, the annealing reaction and the elongation reaction are performed at the same temperature. According to the above-mentioned FIG. 1, from the viewpoint of the activity efficiency of the polymerase, as the second temperature, around 70° C. is most suitable. The type of the liquid 4 is not particularly limited as long as it can be heated to the second temperature by the second hot plate 12, and for example, an aqueous sodium chloride solution and an oil can be exemplified.

The arm 30 is configured such that one end 30a is fixed by the fixing section 32 and the other end 30b is a free end. The end 30b of the arm 30 supports the container 8 containing the reaction solution 6. The arm 30 is operated by a motor (not shown) such that the end 30b reciprocates arcuately while fixing the end 30a.

By the reciprocation of the arm 30, the reaction solution 6 is alternately placed in the liquid 2 heated to the first temperature and in the liquid 4 heated to the second temperature. According to this, thermal cycling for PCR can be performed for the reaction solution 6. The number of cycles of the thermal cycling in this step can be appropriately set by driving and stopping of the motor, and for example, 20 or more and 60 or less. The conveying time of the reaction solution 6 from the liquid 2 to the liquid 4 and the conveying time of the reaction solution 6 from the liquid 4 to the liquid 2 are, for example, about 0.5 seconds.

In the thermal cycling step (Step S3), a heating time for the denaturation reaction per cycle (in the example shown in the drawing, a time in which the reaction solution 6 is placed in the liquid 2) is, for example, 0.3 seconds or more and 5 seconds or less, preferably 0.5 seconds or more and 2 seconds or less. By setting the heating time for the denaturation reaction to 0.3 seconds or more, it is possible to suppress insufficient denaturation due to a too short denaturation reaction time. By setting the heating time for the denaturation reaction to 5 seconds or less, the thermal cycling speed can be increased.

In the thermal cycling step (Step S3), a heating time for the annealing reaction and the elongation reaction per cycle (in the example shown in the drawing, a time in which the reaction solution 6 is placed in the liquid 4) is, for example, 6 seconds or less, preferably 4 seconds or less, more preferably 3 seconds or less, further more preferably 1 second or more and 1.5 seconds or less. By setting the heating time for the annealing reaction and the elongation reaction to 6 seconds or less, the PCR speed can be increased.

In the thermal cycling step (Step S3), a time per cycle of the thermal cycling is 9 seconds or less, preferably 7 seconds or less, more preferably 6 seconds or less. By setting the time per cycle to 9 seconds or less, the thermal cycling speed can be increased. The time per cycle of the thermal cycling includes a time required for the denaturation reaction, the annealing reaction, and the elongation reaction, and the conveying time of the reaction solution for performing these reactions (for example, the conveying time of the reaction solution 6 from the liquid 2 to the liquid 4 and the conveying time of the reaction solution 6 from the liquid 4 to the liquid 2).

Subsequently, the fluorescence intensity of the reaction solution is measured (Step S4). For example, the reaction solution after thermal cycling is performed is transferred to a light transmissive container, and the fluorescence intensity is measured by irradiating the light transmissive container with light. By doing this, the amplification amount of the nucleic acid can be quantitatively determined.

The nucleic acid amplification reaction method according to this embodiment has, for example, the following characteristics.

In the nucleic acid amplification reaction method, the Tm value of the primer is 65° C. or higher and 80° C. or lower, and in the step of performing a reverse transcription reaction (Step S2), a heating time for the reaction solution is 5 seconds or more and 480 seconds or less. Therefore, the nucleic acid amplification reaction method is a nucleic acid amplification reaction method capable of performing a reverse transcription reaction and also increasing the thermal cycling speed, and can suppress amplification of a nonspecific nucleic acid (see the below-mentioned “3. Experimental Examples” for the details).

In the nucleic acid amplification reaction method, in the heating step for performing a reverse transcription reaction (Step S2), the reaction solution may be heated to 50° C. or higher and 70° C. or lower, and a heating time for the reaction solution may be 10 seconds or more and 60 seconds or less. Therefore, according to the nucleic acid amplification reaction method, in the measurement of a fluorescence intensity, the fluorescence intensity can be increased (see the below-mentioned “3. Experimental Examples” for the details).

In the nucleic acid amplification reaction method, the amount of the reverse transcriptase may be 30 units or more and 60 units or less. Therefore, according to the nucleic acid amplification reaction method, in the measurement of a fluorescence intensity, the fluorescence intensity can be increased (see the below-mentioned “3. Experimental Examples” for the details), and also the cost can be reduced.

In the nucleic acid amplification reaction method, in the heating step for performing a reverse transcription reaction (Step S2), the reaction solution may be heated to 60° C. or higher and 70° C. or lower, and a heating time for the reaction solution may be 10 seconds or more and 30 seconds or less. Therefore, according to the nucleic acid amplification reaction method, while ensuring the fluorescence intensity, the reverse transcription reaction time can be reduced (see the below-mentioned “3. Experimental Examples” for the details).

In the nucleic acid amplification reaction method, in the heating step for performing a reverse transcription reaction (Step S2), the reaction solution may be heated to 66° C. or higher and 70° C. or lower, and a heating time for the reaction solution may be 60 seconds or less. Therefore, according to the nucleic acid amplification reaction method, in the measurement of a fluorescence intensity, the fluorescence intensity can be further increased (see the below-mentioned “3. Experimental Examples” for the details).

In the nucleic acid amplification reaction method, in the thermal cycling step (Step S3), the time per cycle of the thermal cycling may be 9 seconds or less. Therefore, according to the nucleic acid amplification reaction method, thermal cycling speed can be increased.

In the nucleic acid amplification reaction method, in the thermal cycling step (Step S3), a heating time for an annealing reaction for the primer may be 6 seconds or less per cycle of the thermal cycling. Therefore, according to the nucleic acid amplification reaction method, thermal cycling speed can be increased.

In the nucleic acid amplification reaction method, the primer may contain an artificial nucleic acid. According to this configuration, amplification of a nonspecific nucleic acid can be suppressed (see the below-mentioned “3. Experimental Examples” for the details).

In the nucleic acid amplification reaction method, the primer may be a sequence-specific primer for a target RNA. According to this configuration, the primer anneals only to a specific base sequence, and therefore, amplification of a nonspecific nucleic acid can be suppressed.

### 3. EXPERIMENTAL EXAMPLES

Hereinafter, the invention will be more specifically described by showing experimental examples. However, the invention is by no means limited to the following experimental examples.

### 3.1. First Experimental Example

### 3.1.1. Preparation of Reaction Solution and Experimental Method

As a template nucleic acid (template RNA), an RNA of type B influenza (InfB) virus was used. The following reaction solution was prepared by adding this template nucleic acid to a reagent.

### Composition of Reaction Solution

| | |
|---|---|
| Platinum Taq polymerase (5 units/μL) | 0.4 μL |
| SuperScript III Reverse Transcriptase (200 units/μL) | 0.04 μL |
| Buffer | 2.0 μL |
| dNTP (10 mM) | 0.25 μL |
| Forward primer for detection of Influenza (100 μM) | 0.32 μL |
| Reverse primer for detection of Influenza (100 μM) | 0.32 μL |
| Fluorescently labeled probe for detection of Influenza (10 μM) | 0.9 μL |
| Influenza RNA (100 copies/μL) | 1.0 μL |
| Distilled water | 4.77 μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

The Tm values and the sequences of the primers and the probe are as shown in the following Table 1.

TABLE 1

| | Tm (° C.) | Sequence |
|---|---|---|
| Forward primer | 76.2 | 5′ TCC TCA ACT CAC TCT TCG AGC GTC TTA |
| | | ATG AAG G 3′ |
| Reverse primer | 77.0 | 5′ CGG TGC TCT TGA CCA AAT TGG GAT AAG |
| | | ACT CC 3′ |
| Probe | 78.3 | 5′ FAM-CCA ATT CGA GCA GCT GAA ACT GCG |
| | | GTG-BHQ1 3′ |

The Tm values shown in Table 1 were calculated according to the above-mentioned formula (1), and the calculation was performed by setting Ct to 500 nM and Na′ to 50 mM in the formula (1). The same applies also to the experimental examples shown below.

10 μL of the reaction solution as described above was placed in a container (Light Cycler Capillaries (20 μL) manufactured by Roche), and a reverse transcription reaction was performed by immersing the container in a water tank heated to 52° C. using a heat block for an arbitrary time. After completion of the reverse transcription reaction, PCR was performed by allowing the container to reciprocate between a high-temperature region (90° C., 2 seconds) and a low-temperature region (66° C., 2 seconds) using the device as shown in FIG. 3. The number of cycles of the thermal cycling was set to 40. In order to activate the polymerase, the reaction solution was initially heated in the high-temperature region for 20 seconds (hot start). Thereafter, the reaction solution was transferred to a different container (MicroAmp Fast Reaction Tubes, manufactured by Applied Biosystems, Inc.), and a fluorescence intensity (endpoint fluorescence intensity) was measured using a Step one Plus Real-time PCR system manufactured by Applied Biosystems, Inc. The above-mentioned experiment was performed twice by changing the heating time for the reaction solution.

### 3.1.2. Results of Measurement of Fluorescence Intensity

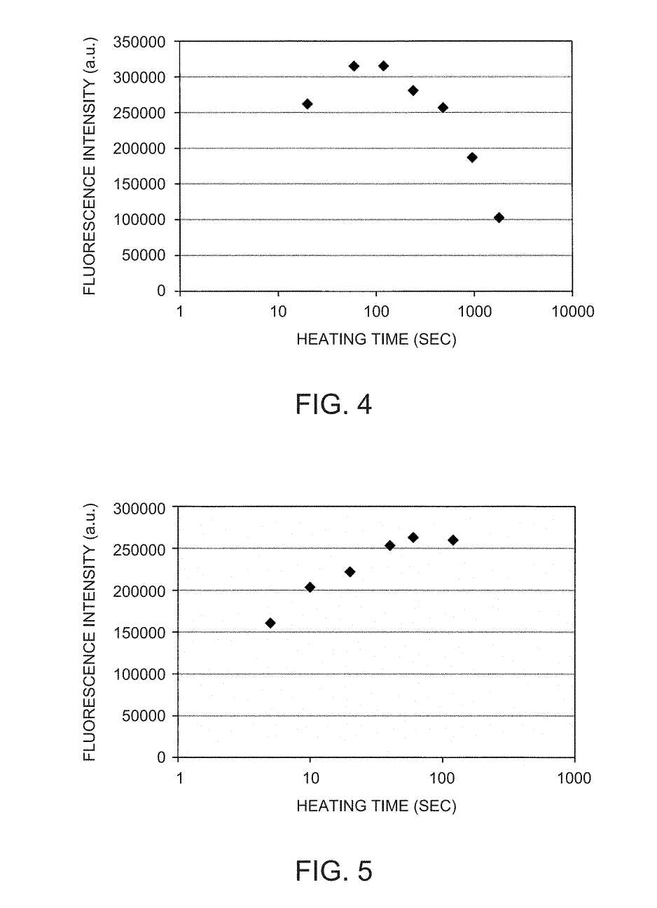

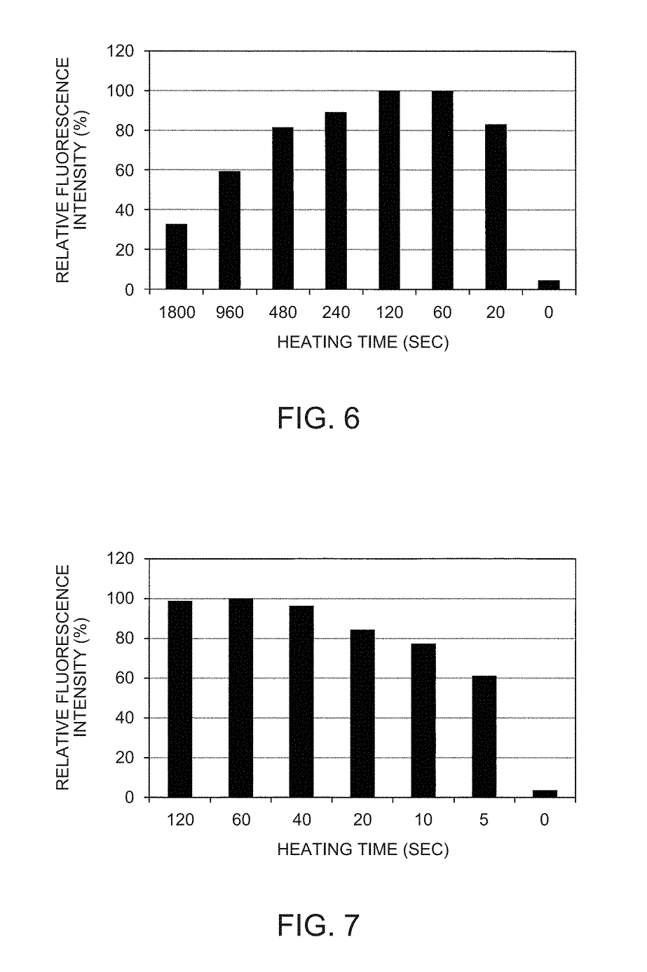

FIGS. 4 and 5 are each a graph showing a relationship between a heating time for a reverse transcription reaction and a fluorescence intensity. FIG. 4 shows the results of the first experiment, and FIG. 5 shows the results of the second experiment. FIG. 6 is a graph showing a relative fluorescence intensity when the fluorescence intensity in the case where the heating time was 60 seconds in FIG. 4 was assumed to be 100%. FIG. 7 is a graph showing a relative fluorescence intensity when the fluorescence intensity in the case where the reverse transcription reaction time was 60 seconds in FIG. 5 was assumed to be 100%.

From FIGS. 6 and 7, it was found that when the heating time for the reverse transcription reaction is set to 5 seconds or more and 480 seconds or less, the relative fluorescence intensity is 60% or more, and the fluorescence intensity can be increased. Further, by setting the heating time for the reverse transcription reaction to 10 seconds or more and 480 seconds or less, the relative fluorescence intensity could be increased to 70% or more, by setting the heating time for the reverse transcription reaction to 20 seconds or more and 480 seconds or less, the relative fluorescence intensity could be increased to 80% or more, and by setting the heating time for the reverse transcription reaction to 40 seconds or more and 120 seconds or less, the relative fluorescence intensity could be increased to 90% or more.

### 3.1.3. Results of Electrophoresis

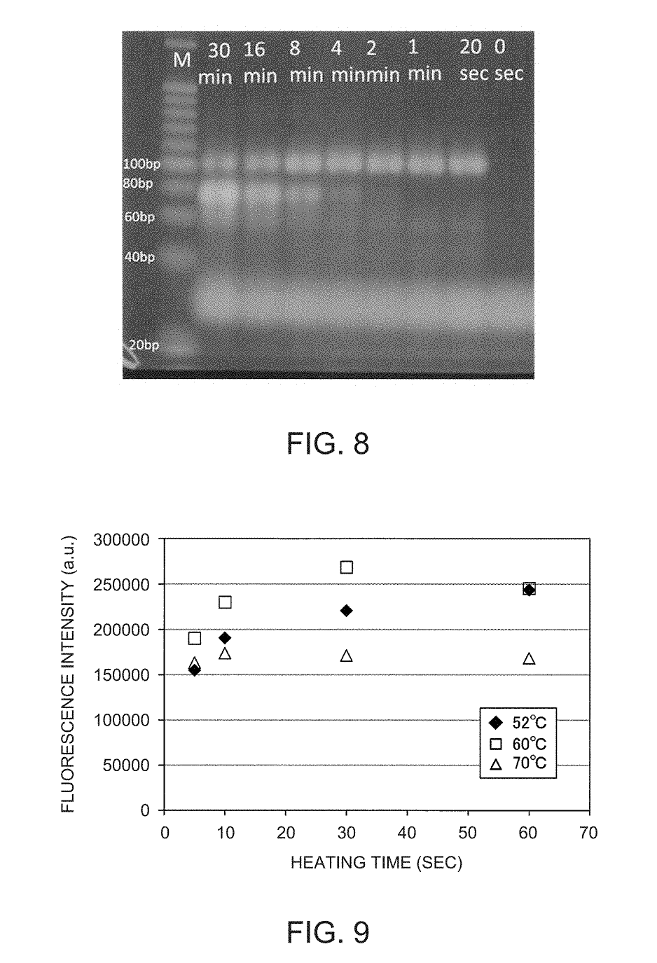

The above reaction solution was analyzed by agarose gel electrophoresis. FIG. 8 shows the results of electrophoresis. In FIG. 8, “M” shows a molecular weight marker.

As shown in FIG. 8, when the heating time for the reverse transcription reaction was long, bands (40 bp or more and 80 bp or less) derived from amplification of a nonspecific nucleic acid were confirmed, and when the heating time was longer than 8 minutes (480 seconds), the bands were significantly confirmed. Further, when the heating time for the reverse transcription reaction was long, a band (103 bp) derived from the target nucleic acid appeared weak, and as the heating time was reduced, the band appeared stronger. Incidentally, when the heating time was 0 sec, the band (103 bp) was not confirmed.

Based on the above results, it was found that when the heating time for the reverse transcription reaction is too long, the primer anneals to a region other than a target region and a nucleic acid is amplified, and amplification of the target nucleic acid is inhibited. This inhibition can be improved by reducing the heating time.

The reason for this is considered as follows. In the case of a usual PCR condition (for example, a condition in which the time for the annealing reaction and the elongation reaction is longer than 2 seconds), the heating time for the reverse transcription reaction is generally 30 minutes to 1 hour. However, in the case of a high-speed thermal cycling condition as in the first experimental example (for example, a condition in which the time for the annealing reaction and the elongation reaction is 2 seconds or less), a primer having a high Tm value such that the Tm value is 65° C. or higher and 80° C. or lower is used for increasing the thermal cycling speed. It is considered that in such a primer, for example, the number of bases of the primer is large, and when the heating time for the reverse transcription reaction is long, the primer anneals to a region other than a target region, and amplification of a nonspecific nucleic acid is likely to occur. Therefore, it is considered that under a high-speed thermal cycling condition, when the heating time was set to 480 seconds or less, amplification of a nonspecific nucleic acid could be suppressed. This presumption is one hypothesis.

### 3.2. Second Experimental Example

A fluorescence intensity was measured in the same manner as in the first experimental example except that the heating temperature and the heating time for the reverse transcription reaction were changed. The heating temperature for the reverse transcription reaction was changed to 52° C., 60° C., and 70° C. FIG. 9 is a graph showing a relationship between the heating time for the reverse transcription reaction and the fluorescence intensity.

As shown in FIG. 9, it was found that by setting the heating temperature to 50° C. or higher and 70° C. or lower, and the heating time to 10 seconds or more and 60 seconds or less, the fluorescence intensity can be increased as compared with the case where the heating time is set to, for example, 5 seconds or less.

It was found that in the case where the heating temperature was set to 60° C., the fluorescence intensity was higher than in the case where the heating temperature was set to 52° C., and the heating time can be reduced without decreasing the fluorescence intensity. Further, in the case where the heating temperature was set to 70° C., when the heating time was set to 30 seconds or more, the fluorescence intensity was decreased as compared with the case where the heating temperature was set to 52° C. and 60° C., however, when the heating time was set to 10 seconds, the fluorescence intensity was almost the same as in the case where the heating temperature was set to 52° C. In the second experimental example, the optimal condition was as follows: the heating temperature is 60° C. and the heating time is 30 seconds. Therefore, it was found that by setting the heating temperature to 60° C. or higher and 70° C. or lower, and the heating time to 10 seconds or more and 60 seconds or less, the reverse transcription reaction time can be reduced while ensuring the fluorescence intensity.

### 3.3. Third Experimental Example

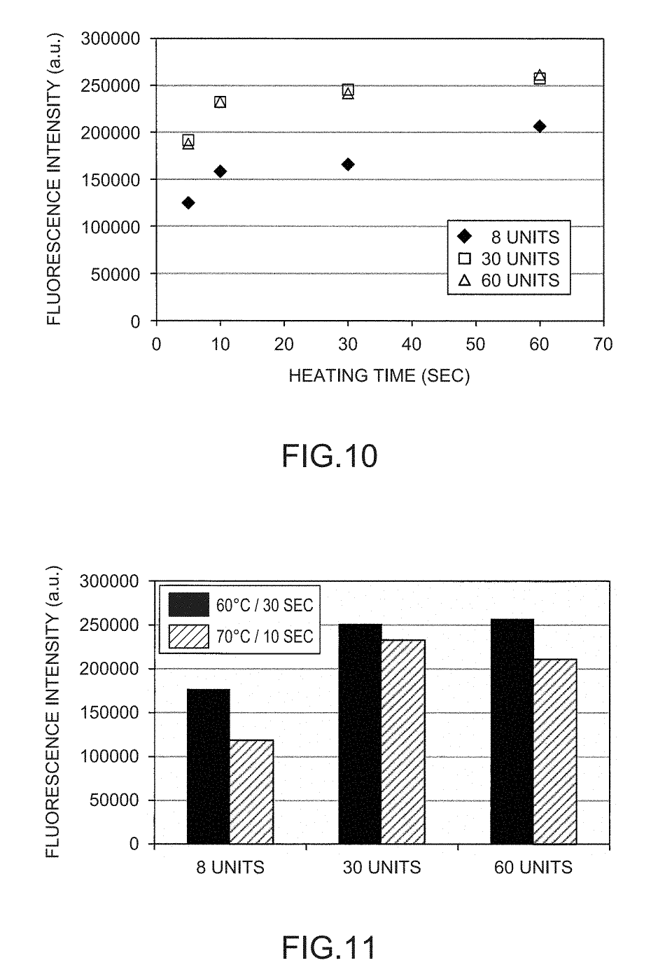

A fluorescence intensity was measured in the same manner as in the first experimental example except that the heating time for the reverse transcription reaction and the amount of the reverse transcriptase were changed. The amount of the reverse transcriptase was changed to 8 units, 30 units, and 60 units. FIG. 10 is a graph showing a relationship between the heating time for the reverse transcription reaction and the fluorescence intensity.

As shown in FIG. 10, it was found that by setting the amount of the reverse transcriptase to 30 units or more and 60 units or less, the fluorescence intensity can be increased as compared with the case where the amount of the reverse transcriptase is set to, for example, 8 units.

### 3.4. Fourth Experimental Example

A fluorescence intensity was measured in the same manner as in the first experimental example except that the heating temperature and the heating time for the reverse transcription reaction and the amount of the reverse transcriptase were changed. Specifically, according to the second experimental example, in the case where the heating temperature for the reverse transcription reaction was set to 60° C., the fluorescence intensity was highest when the heating time was set to 30 seconds, and in the case where the heating temperature for the reverse transcription reaction was set to 70° C., the fluorescence intensity was highest when the heating time was set to 10 seconds. Therefore, in the fourth experimental example, in the case where the heating temperature was set to 60° C. and the heating time was set to 30 seconds (60° C./30 sec) and in the case where the heating temperature was set to 70° C. and the heating time was set to 10 seconds (70° C./10 sec), an experiment was performed by changing the amount of the reverse transcriptase to 8 units, 30 units, and 60 units. FIG. 11 is a graph showing a relationship between the amount of the reverse transcriptase and the fluorescence intensity.

As shown in FIG. 11, by setting the amount of the reverse transcriptase to 30 units or more, the fluorescence intensity could be increased, and in the case of the “70° C./10 sec”, although the heating time was as short as 10 seconds, almost the same fluorescence intensity as in the case of the “60° C./30 sec” could be obtained.

### 3.5. Fifth Experimental Example

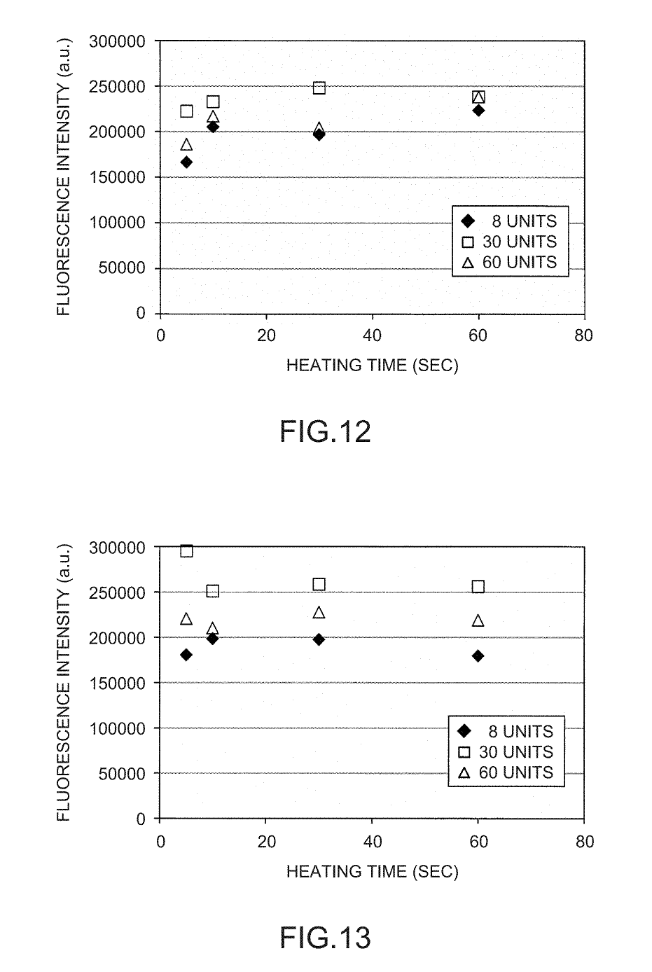

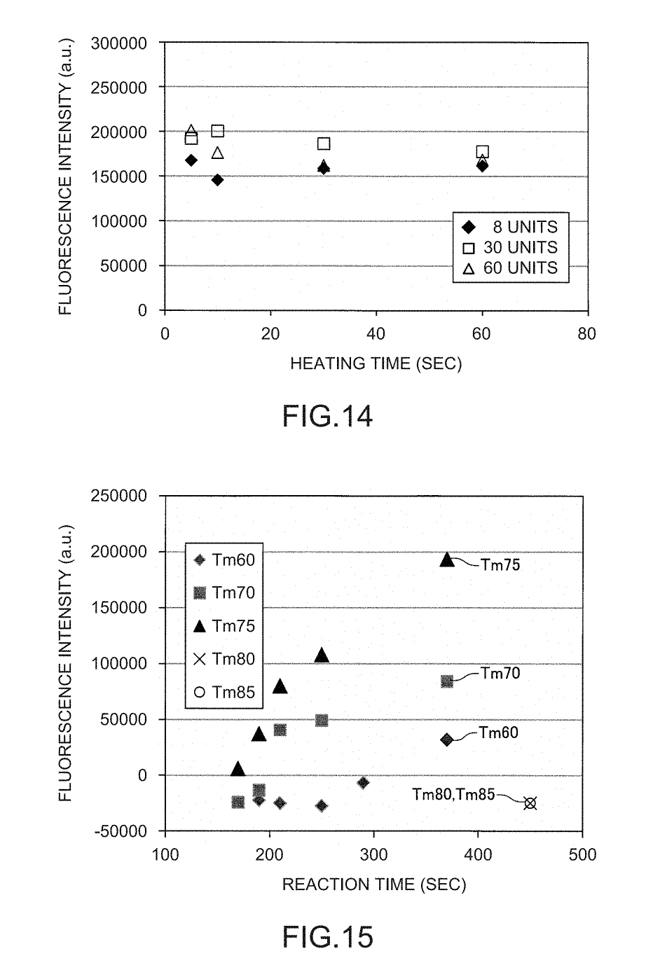

According to the fourth experimental example, it was found that even if the temperature is increased, by increasing the amount of the reverse transcriptase, the reverse transcription reaction can be performed in a short time without decreasing the fluorescence intensity. Therefore, in the fifth experimental example, under the condition that the heating temperature was set to a temperature higher than 60° C. (66° C., 70° C., and 74° C.), an experiment was performed by changing the heating time for the reverse transcription reaction, and further changing the amount of the reverse transcriptase to 8 units, 30 units, and 60 units. A fluorescence intensity was measured in the same manner as in the first experimental example except that the heating temperature and the heating time for the reverse transcription reaction and the amount of the reverse transcriptase were changed. FIGS. 12 to 14 are each a graph showing a relationship between the heating time for the reverse transcription reaction and the fluorescence intensity. FIG. 12 shows the case where the heating temperature for the reverse transcription reaction was set to 66° C., FIG. 13 shows the case where the heating temperature for the reverse transcription reaction was set to 70° C., and FIG. 14 shows the case where the heating temperature for the reverse transcription reaction was set to 74° C.

As shown in FIGS. 12 to 14, in the case where the heating temperature was set to 74° C., the fluorescence intensity was decreased as compared with the case where the heating temperature was set to 66° C. and 70° C. Therefore, it was found that by setting the heating time to 60 seconds or less and the heating temperature to 66° C. or higher and 70° C. or lower, the fluorescence intensity can be increased. Further, also in the fifth experimental example, in the same manner as in the third experimental example, in the case where the amount of the reverse transcriptase was set to 30 units and 60 units, the fluorescence intensity was higher than in the case where the amount of the reverse transcriptase was set to 8 units.

### 3.6. Sixth Experimental Example

### 3.6.1. Preparation of Reaction Solution and Experimental Method

As a template nucleic acid (template DNA), a Mycoplasma species DNA was used. The following reaction solution was prepared by adding this template nucleic acid to a reagent.

### Composition of Reaction Solution

| | |
|---|---|
| Platinum Tag polymerase (5 units/μL) | 0.4 μL |
| Buffer | 2.0 μL |
| dNTP (10 mM) | 0.25 μL |
| Forward primer for detection of Mycoplasma species (20 μM) | 1.2 μL |
| Reverse primer for detection of Mycoplasma species (20 μM) | 1.2 μL |
| Fluorescently labeled probe for detection of Mycoplasma | 0.9 μL |
| species (10 μM) | |
| Mycoplasma species DNA (100 copies/μL) | 1.0 μL |
| Distilled water | 3.05 μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

In this experiment, primers having a different Tm value were used. Specifically, primers having a Tm value of about 60° C. (Tm60), about 70° C. (Tm70), about 75° C. (Tm75), about 80° C. (Tm80), or about 85° C. (Tm85) were used. The Tm values and the sequences of the primers, and the sequence of the probe are as shown in the following Table 2.

TABLE 2

| | | Tm (° C.) | Sequence |
|---|---|---|---|
| Tm 60 | Forward | 62.6 | 5′ AAA TCC AGG TAC GGG TGA AG 3′ |
| | primer | | |
| | Reverse | 60.6 | 5′ GTC CTG ATC AAT ATT AAG CTA CAG TAA A 3′ |
| | primer | | |
| Tm 70 | Forward | 70.4 | 5′ AAA TCC AGG TAC GGG TGA AGA CAC C 3′ |
| | primer | | |
| | Reverse | 70.7 | 5′ GTC CTG ATC AAT ATT AAG CTA CAG TAA AGC |
| | primer | | TTC ACG 3′ |
| Tm 75 | Forward | 75.9 | 5′ GGT GAA ATC CAG GTA CGG GTG AAG ACA CC 3′ |
| | primer | | |
| | Reverse | 75.4 | 5′ GTC CTG ATC AAT ATT AAG CTA CAG TAA AGC |
| | primer | | TTC ACG GGG 3′ |
| Tm 80 | Forward | 80.2 | 5′ GGT GAA ATC CAG GTA CGG GTG AAG ACA CCC |
| | primer | | G 3′ |
| | Reverse | 79.0 | 5′ CAT GAT AAT GTC CTG ATC AAT ATT AAG CTA |
| | primer | | CAG TAA AGC TTC ACG GGG TC 3′ |
| Tm 85 | Forward | 85.5 | 5′ GGT GAA ATC CAG GTA CGG GTG AAG ACA CCC |
| | primer | | GTT AGG CGC 3′ |
| | Reverse | 84.9 | 5′ GCA TCG ATT GCT CCT ACC TAT TCT CTA CAT |
| | primer | | GAT AAT GTC CTG ATC AAT ATT AAG CTA CAG TAA |
| | | | AGC TTC ACG GGG TC 3′ |

| | |
|---|---|
| Probe | 5′ FAM-CGG GAC GGA AAG ACC-NFQ-MGB 3′ |

10 μL of the reaction solution as described above was placed in a container (Light Cycler Capillaries (20 μL) manufactured by Roche), and PCR was performed by allowing the container to reciprocate between a high-temperature region and a low-temperature region using the device as shown in FIG. 3. The number of cycles of the thermal cycling was set to 40. Thereafter, the reaction solution was transferred to a different container (MicroAmp Fast Reaction Tubes, manufactured by Applied Biosystems, Inc.), and a fluorescence intensity was measured using a Step one Plus Real-time PCR system manufactured by Applied Biosystems, Inc.

In the PCR using each of the primers (Tm60, Tm70, Tm75, Tm80, and Tm85), the heating temperature (high temperature) for the denaturation reaction was set to 87° C. In the PCR using Tm60, Tm70, Tm75, Tm80, and Tm85, the heating temperature (low temperature) for the annealing reaction and the elongation reaction was set to 60° C., 63° C., 66° C., 69° C., and 72° C., respectively. In the PCR, the heating time (the time at the high temperature) for the denaturation reaction per cycle, the heating time (the time at the low temperature) for the annealing reaction and the elongation reaction per cycle, and the reaction time are shown in the following Table 3. Incidentally, in order to activate the polymerase, the reaction solution was initially heated to the high temperature for 10 seconds (hot start). The reaction time is obtained by, in addition to the polymerase activation time, adding the time at the high temperature and the time at the low temperature multiplied by 40 (the number of cycles), and further adding the conveying time of the reaction solution. Further, in Table 3, the time per cycle of the thermal cycling is obtained by adding the conveying time from the high-temperature region to the low-temperature region (0.5 sec) and the conveying time from the low-temperature region to the high-temperature region (0.5 sec) to the sum of the time at the high temperature and the time at the low temperature. For example, in the case where the reaction time is 370 seconds, the time per cycle of the thermal cycling is as follows: the time at the high temperature (2 sec)+the time at the low temperature (6 sec)+the conveying time from the high-temperature region to the low-temperature region (0.5 sec)+the conveying time from the low-temperature region to the high-temperature region (0.5 sec)=9 sec.

TABLE 3

| | | | | | | | |
|---|---|---|---|---|---|---|---|
| Time at high | 2 | 2 | 2 | 2 | 2 | 2 | 4 |
| temperature (sec) | | | | | | | |
| Time at low | 1 | 1.5 | 2 | 3 | 4 | 6 | 6 |
| temperature (sec) | | | | | | | |
| Reaction time (sec) | 170 | 190 | 210 | 250 | 290 | 370 | 450 |

### 3.6.2. Results of Measurement of Fluorescence Intensity

FIG. 15 is a graph showing a relationship between a PCR reaction time and a fluorescence intensity. As shown in FIG. 15, in the case where the time at the low temperature was 6 seconds (in the case where the time per cycle was 9 seconds), amplification was confirmed when using Tm70 and Tm75, however, in the case where the time at the low temperature was 4 seconds (in the case where the time per cycle was 7 seconds) or less, amplification of a nucleic acid was not confirmed when using Tm60, and amplification was confirmed when using Tm70 and Tm75. Therefore, it was found that by setting the Tm value of the primer to 70° C. or higher and lower than 80° C., even in the case of high-speed PCR in which the time at the low temperature is 4 seconds or less, a nucleic acid can be amplified. This is considered to be because a primer having a higher Tm value anneals to a template nucleic acid faster, and therefore, Tm70 and Tm75 are more suitable for increasing the thermal cycling speed than Tm60. Further, according to the above-mentioned FIG. 1, it is considered that Tm70 and Tm75 have a higher polymerase activity efficiency than Tm60 and can accelerate the elongation reaction, and therefore, Tm70 and Tm75 are more suitable for increasing the thermal cycling speed than Tm60. When considering a variation in the device used in this experimental example, it can be said that when the fluorescence intensity is 35000 or more, a nucleic acid is reliably amplified. Therefore, in the case where the time per cycle is 9 seconds, it cannot be said that a nucleic acid is reliably amplified when using Tm60, and it can be said that a nucleic acid is reliably amplified when using Tm70 and Tm75.

Further, from FIG. 15, it was found that even if the time at the low temperature is 2 seconds or less, when the Tm value of a primer is 70° C. or higher and 75° C. or lower, a nucleic acid can be amplified. Further, in FIG. 15, when using Tm80 and Tm85, amplification of a nucleic acid was not confirmed. This is considered to be because the Tm value was too high, and therefore, a primer dimer or the like was formed.

In FIG. 15, a value obtained by subtracting the fluorescence intensity of the unreacted solution for which thermal cycling was not performed (background) from the endpoint fluorescence intensity is plotted. The plot in which the fluorescence intensity shows a negative value is considered to be a measurement error.

### 3.7. Seventh Experimental Example

### 3.7.1. Preparation of Reaction Solution and Experimental Method

As a template nucleic acid (template DNA), a Bordetella pertussis DNA was used. The following reaction solution was prepared by adding this template nucleic acid to a reagent.

### Composition of Reaction Solution

| | |
|---|---|
| Platinum Tag polymerase (5 units/μL) | 0.4 μL |
| Buffer | 2.0 μL |
| dNTP (10 mM) | 0.25 μL |
| Forward primer for detection of Bordetella pertussis (100 μM) | 0.32 μL |
| Reverse primer for detection of Bordetella pertussis (100 μM) | 0.32 μL |
| Fluorescently labeled probe for detection of Bordetella | 0.9 μL |
| pertussis (10 μM) | |
| Bordetella pertussis DNA (20 copies or 100 copies/μL) | 1.0 μL |
| Distilled water | 4.81 μL |

As the fluorescently labeled probe, TaqMan (registered trademark) probe manufactured by Sigma-Aldrich Co. LLC. was used.

The buffer (buffer solution) contains MgCl₂, Tris-HCl (pH 9.0), and KCl. The concentration of MgCl₂ contained in the reaction solution was set to 5 mM.

The Tm values and the sequences of the primers, and the sequence of the probe are as shown in the following Table 4.

TABLE 4

| | Tm (° C.) | Sequence |
|---|---|---|
| Forward | 80.8 | 5′ ATC AAG CAC CGC TTT ACC CGA |
| primer | | CCT TAC CGC C 3′ |
| Reverse | 80.3 | 5′ TTG G GA GTT CTG GTA GGT |
| primer | | GTG AGC GTA AGC CCA 3′ |
| Probe | | 5′ FAM-AAT GGC AAG GCC GAA CGC |
| | | TTC A-NFQ-MGB 3′ |

PCR was performed for 10 μL of the reaction solution as described above by performing hot start for 10 seconds in the same manner as in the sixth experimental example. The high temperature was set to 90° C., and the low temperature was set to 60° C. The heating time (the time at the high temperature) for the denaturation reaction per cycle, the heating time (the time at the low temperature) for the annealing reaction and the elongation reaction per cycle, and the reaction time are shown in the following Table 5.

TABLE 5

| | | | | | |
|---|---|---|---|---|---|
| Time at high temperature (sec) | 1 | 2 | 2 | 2 | 2 |
| Time at low temperature (sec) | 1 | 1 | 2 | 3 | 4 |
| Reaction time (sec) | 130 | 170 | 210 | 250 | 290 |

### 3.7.2. Results of Measurement of Fluorescence Intensity

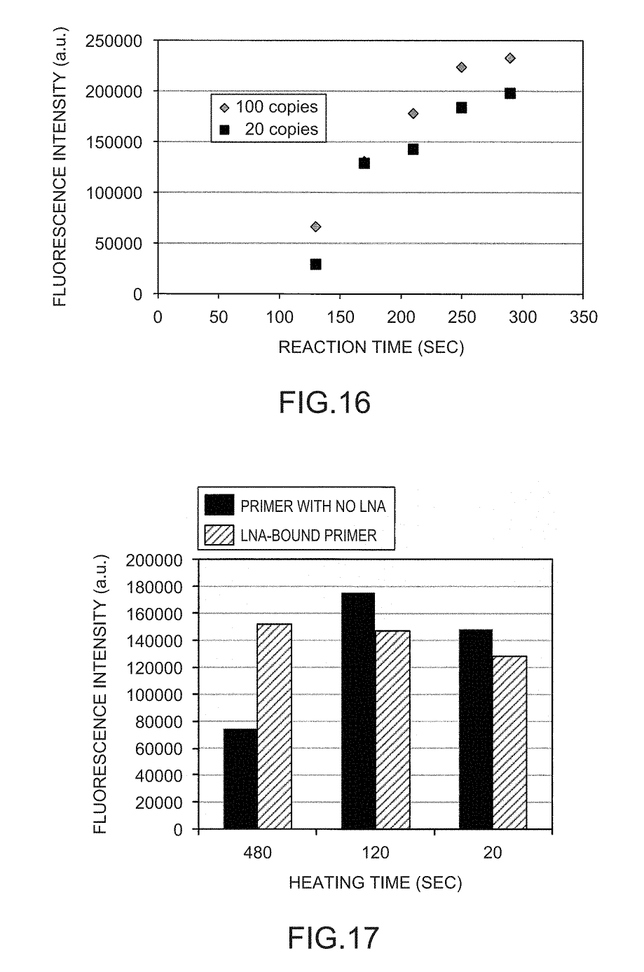

FIG. 16 is a graph showing a relationship between a PCR reaction time and a fluorescence intensity. As shown in FIG. 16, even if the Tm value of the primer was about 80° C., amplification of a nucleic acid could be confirmed.

### 3.8. Eighth Experimental Example

### 3.8.1. Preparation of Reaction Solution and Experimental Method

A fluorescence intensity was measured in the same manner as in the first experimental example except that in place of the reverse primer shown in Table 1, an LNA-bound primer (a primer having an LNA bound thereto (inserted therein)) shown in the following Table 6 as a reverse primer for InfB was used, and the time for the reverse transcription reaction for the reaction solution was changed to 20 seconds, 120 seconds, and 480 seconds.

TABLE 6

| | Tm (° C.) | Sequence |
|---|---|---|
| Reverse | 77.1 | 5′ CGG TGC TCT TGA CCA AAT |
| primer | | TGG 3′ |

In Table 6, a base having an LNA bound thereto is underlined. That is, an LNA is bound to “T” which is a seventh base from the 5′-end side and “C” which is the 13^{th} base from the 5′-end side.

### 3.8.2. Results of Measurement of Fluorescence Intensity

FIG. 17 is a graph showing a reverse transcription reaction time and a fluorescence intensity. In FIG. 17, the fluorescence intensity in the case where the primer used in the first experimental example (a primer with no LNA, that is, a primer having no LNA bound thereto) was used, and the fluorescence intensity in the case where the primer shown in Table 6 (an LNA-bound primer) was used are shown.

As shown in FIG. 17, it was found that by using the LNA-bound primer, even if reverse transcription is performed for 480 seconds, the fluorescence intensity can be maintained high without decreasing the fluorescence intensity.

### 3.8.3. Results of Electrophoresis

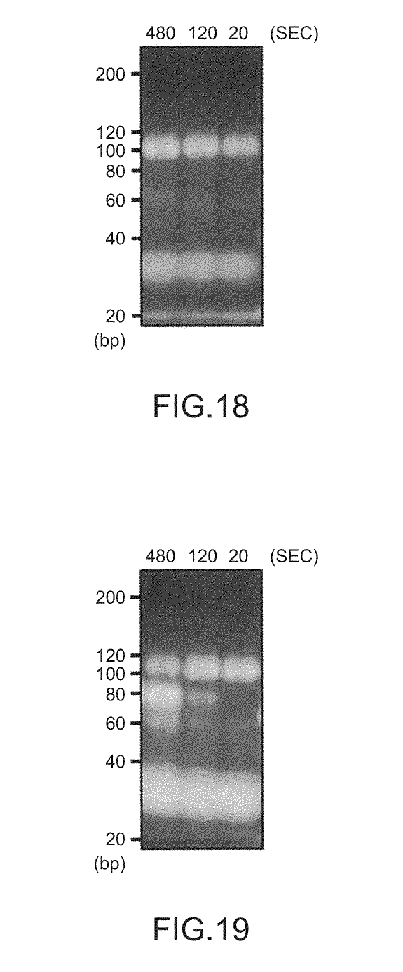

The above reaction solution was analyzed by agarose gel electrophoresis. FIG. 18 shows the results of electrophoresis in the case where the LNA-bound primer was used. FIG. 19 shows the results of electrophoresis in the case where the primer with no LNA was used.

As shown in FIG. 19, in the case where the primer with no LNA was used, when the heating time for the reverse transcription reaction was long, bands (40 bp or more and 90 bp or less) derived from amplification of a nonspecific nucleic acid were confirmed. On the other hand, as shown in FIG. 18, in the case where the LNA-bound primer was used, even if the heating time for the reverse transcription reaction was longer than 120 seconds, nonspecific amplification could not be confirmed. Further, in the case where the primer with no LNA was used, when the heating time for the reverse transcription reaction was long, a band (103 bp) derived from the target nucleic acid appeared weak, and as the heating time was reduced, the band appeared stronger. On the other hand, in the case where the LNA-bound primer was used, a band (103 bp) derived from the target nucleic acid did not appear weaker even if the heating time for the reverse transcription reaction was 480 seconds. Incidentally, the bands of 20 bp or more and 40 bp or less are derived from the primer.

Based on the above results, it was found that when the LNA-bound primer is used, nonspecific amplification due to a long heating time for the reverse transcription reaction can be suppressed, and amplification of a target nucleic acid is not inhibited. Therefore, it was found that by using the LNA-bound primer, the robustness of the nucleic acid amplification method can be improved, and stable RT (Reverse Transcription)-PCR can be performed.

As the interpretation of this result, the following speculation can be made. It is considered that when the primer is long, the primer anneals to a region other than the target region and a nucleic acid is amplified, and therefore, amplification of the target nucleic acid is inhibited. That is, it is considered that by using a primer having a short chain length, such a problem can be eliminated. However, in such a case, even if the length of the primer is merely reduced, the Tm value is decreased, and therefore, high-speed PCR cannot be realized. In this experimental example, an LNA was bound to the primer, and the primer having a high Tm value while reducing the chain length of the primer was used, whereby high-speed PCR could be realized while imparting robustness to reverse transcription.

The invention includes substantially the same configurations (for example, configurations having the same functions, methods, and results, or configurations having the same objects and effects) as the configurations described in the embodiments. Further, the invention includes configurations in which a part that is not essential in the configurations described in the embodiments is substituted. Further, the invention includes configurations having the same effects as in the configurations described in the embodiments, or configurations capable of achieving the same objects as in the configurations described in the embodiments. In addition, the invention includes configurations in which known techniques are added to the configurations described in the embodiments.

The entire disclosure of Japanese Patent Application Nos. 2016-149430, filed Jul. 29, 2016 and 2017-049610, filed Mar. 15, 2017 are expressly incorporated by reference herein.

### Sequence Listing Free Text

SEQ ID NO: 1 is the sequence of a forward primer for InfB.

SEQ ID NO: 2 is the sequence of a reverse primer for InfB.

SEQ ID NO: 3 is the sequence of a fluorescently labeled probe for InfB.

SEQ ID NO: 4 is the sequence of a forward primer for Mycoplasma bacteria.

SEQ ID NO: 5 is the sequence of a reverse primer for Mycoplasma bacteria.

SEQ ID NO: 6 is the sequence of a forward primer for Mycoplasma bacteria.

SEQ ID NO: 7 is the sequence of a reverse primer for Mycoplasma bacteria.

SEQ ID NO: 8 is the sequence of a forward primer for Mycoplasma bacteria.

SEQ ID NO: 9 is the sequence of a reverse primer for Mycoplasma bacteria.

SEQ ID NO: 10 is the sequence of a forward primer for Mycoplasma bacteria.

SEQ ID NO: 11 is the sequence of a reverse primer for Mycoplasma bacteria.

SEQ ID NO: 12 is the sequence of a forward primer for Mycoplasma bacteria.

SEQ ID NO: 13 is the sequence of a reverse primer for Mycoplasma bacteria.

What is claimed is:

1. A nucleic acid amplification reaction method, comprising:
a heating step of heating a reaction solution containing a reverse transcriptase, a polymerase, a primer, and a probe for performing a reverse transcription reaction; and
a thermal cycling step of performing thermal cycling for amplifying a nucleic acid for the reaction solution after the heating step, wherein
the Tm value of the primer is 65° C. or higher and 80° C. or lower, and
in the heating step,
a heating time for the reaction solution is 5 seconds or more and 480 seconds or less.

2. The nucleic acid amplification reaction method according to claim 1, wherein in the heating step, the reaction solution is heated to 50° C. or higher and 70° C. or lower.

3. The nucleic acid amplification reaction method according to claim 2, wherein in the heating step, a heating time for the reaction solution is 10 seconds or more and 60 seconds or less.

4. The nucleic acid amplification reaction method according to claim 1, wherein the amount of the reverse transcriptase contained in the reaction solution is 30 units or more and 60 units or less.

5. The nucleic acid amplification reaction method according to claim 4, wherein in the heating step,
the reaction solution is heated to 60° C. or higher and 70° C. or lower, and
a heating time for the reaction solution is 10 seconds or more and 30 seconds or less.

6. The nucleic acid amplification reaction method according to claim 1, wherein in the heating step,
the reaction solution is heated to 66° C. or higher and 70° C. or lower, and
a heating time for the reaction solution is 60 seconds or less.

7. The nucleic acid amplification reaction method according to claim 1, wherein in the thermal cycling step, the time per cycle of the thermal cycling is 9 seconds or less.

8. The nucleic acid amplification reaction method according to claim 7, wherein a heating time for an annealing reaction for the primer is 6 seconds or less.

9. The nucleic acid amplification reaction method according to claim 1, wherein
the reaction solution contains a divalent cation, and
the concentration of the divalent cation contained in the reaction solution is 2 mM or more and 7.5 mM or less.

10. The nucleic acid amplification reaction method according to claim 9, wherein
the reaction solution contains MgCl₂,
the divalent cation is derived from MgCl₂, and
the concentration of MgCl₂ contained in the reaction solution is 4 mM or more and 7.5 mM or less.

11. The nucleic acid amplification reaction method according to claim 9, wherein
the reaction solution contains MgSO₄,
the divalent cation is derived from MgSO₄, and
the concentration of MgSO₄ contained in the reaction solution is 2 mM or more and 3 mM or less.

12. The nucleic acid amplification reaction method according to claim 1, wherein the probe is a hydrolysis probe.

13. The nucleic acid amplification reaction method according to claim 1, wherein the probe contains at least one of an artificial nucleic acid and a minor groove binder molecule.

14. The nucleic acid amplification reaction method according to claim 1, wherein the reverse transcriptase is derived from mouse Moloney murine leukemia virus.

15. The nucleic acid amplification reaction method according to claim 1, wherein the primer contains an artificial nucleic acid.

16. The nucleic acid amplification reaction method according to claim 1, wherein the primer is a sequence-specific primer for a target RNA.

17. A reagent, which is a reagent for performing a reverse transcription reaction and a nucleic acid amplification reaction, comprising a reverse transcriptase, a polymerase, a primer, a probe, and MgCl₂, wherein
the Tm value of the primer is 65° C. or higher and 80° C. or lower, and
when the reagent becomes a reaction solution for performing a reverse transcription reaction and a nucleic acid amplification reaction, the concentration of MgCl₂ contained in the reaction solution is 4 mM or more and 7.5 mM or less.

18. A reagent, which is a reagent for performing a reverse transcription reaction and a nucleic acid amplification reaction, comprising a reverse transcriptase, a polymerase, a primer, a probe, and MgSO₄, wherein
the Tm value of the primer is 65° C. or higher and 80° C. or lower, and
when the reagent becomes a reaction solution for performing a reverse transcription reaction and a nucleic acid amplification reaction, the concentration of MgSO₄ contained in the reaction solution is 2 mM or more and 3 mM or less.

19. A method of using the reagent according to claim 17, comprising preparing the reaction solution by bringing the reagent and a template nucleic acid solution containing a template nucleic acid into contact with each other;
performing a reverse transcription reaction by heating the reaction solution for 5 seconds or more and 480 seconds or less; and
performing, after the performing of the reverse transcription reaction, performing a nucleic acid amplification reaction by performing thermal cycling for the reaction solution.

20. A method of using the reagent according to claim 19, comprising preparing the reaction solution by bringing the reagent and a template nucleic acid solution containing a template nucleic acid into contact with each other;
performing a reverse transcription reaction by heating the reaction solution for 5 seconds or more and 480 seconds or less; and
performing, after the performing of the reverse transcription reaction, performing a nucleic acid amplification reaction by performing thermal cycling for the reaction solution.


SEQUENCE LISTING (18 sequences)

SEQ ID NO: 1 - DNA, 34 bp, Artificial Sequence
  InfB Forward primer
tcctcaactcactcttcgagcgtcttaatgaagg

SEQ ID NO: 2 - DNA, 32 bp, Artificial Sequence
  InfB Reverse primer
cggtgctcttgaccaaattgggataagactcc

SEQ ID NO: 3 - DNA, 27 bp, Artificial Sequence
  InfB Fluorescent probe
ccaattcgagcagctgaaactgcggtg

SEQ ID NO: 4 - DNA, 20 bp, Artificial Sequence
  Mycoplasma bacteria Forward primer
aaatccaggtacgggtgaag

SEQ ID NO: 5 - DNA, 28 bp, Artificial Sequence
  Mycoplasma bacteria Reverse primer
gtcctgatcaatattaagctacagtaaa

SEQ ID NO: 6 - DNA, 25 bp, Artificial Sequence
  Mycoplasma bacteria Forward primer
aaatccaggtacgggtgaagacacc

SEQ ID NO: 7 - DNA, 36 bp, Artificial Sequence
  Mycoplasma bacteria Reverse primer
gtcctgatcaatattaagctacagtaaagcttcacg

SEQ ID NO: 8 - DNA, 29 bp, Artificial Sequence
  Mycoplasma bacteria Forward primer
ggtgaaatccaggtacgggtgaagacacc

SEQ ID NO: 9 - DNA, 39 bp, Artificial Sequence
  Mycoplasma bacteria Reverse primer
gtcctgatcaatattaagctacagtaaagcttcacgggg

SEQ ID NO: 10 - DNA, 31 bp, Artificial Sequence
  Mycoplasma bacteria Forward primer
ggtgaaatccaggtacgggtgaagacacccg

SEQ ID NO: 11 - DNA, 50 bp, Artificial Sequence
  Mycoplasma bacteria Reverse primer
catgataatgtcctgatcaatattaagctacagtaaagcttcacggggtc

SEQ ID NO: 12 - DNA, 39 bp, Artificial Sequence
  Mycoplasma bacteria Forward primer
ggtgaaatccaggtacgggtgaagacacccgttaggcgc

SEQ ID NO: 13 - DNA, 77 bp, Artificial Sequence
  Mycoplasma bacteria Reverse primer
gcatcgattgctcctacctattctctacatgataatgtcctgatcaatattaagctacagtaaagcttcacggggtc

SEQ ID NO: 14 - DNA, 15 bp, Artificial Sequence
  Mycoplasma bacteria Fluorescent probe
cgggacggaaagacc

SEQ ID NO: 15 - DNA, 31 bp, Artificial Sequence
  Bordetella pertussis Forward primer
atcaagcaccgctttacccgaccttaccgcc

SEQ ID NO: 16 - DNA, 33 bp, Artificial Sequence
  Bordetella pertussis Reverse primer
ttgggagttctggtaggtgtgagcgtaagccca

SEQ ID NO: 17 - DNA, 22 bp, Artificial Sequence
  Bordetella pertussis Fluorescent probe
aatggcaaggccgaacgcttca

SEQ ID NO: 18 - DNA, 21 bp, Artificial Sequence
  InfB Reverse primer
cggtgctcttgaccaaattgg